(19) United States
(12) Patent Application Publication
(10) Pub. No.: US 2001/0021704 A1
(43) Pub. Date: Sep. 13, 2001

(54) Pharmaceutical compositions for the treatment of tumor diseases

(76) Inventors: Miklos Ghyczy, Koln (DE); Jorg Hager, Koln (DE); Armin Wendel, Koln (DE)

Correspondence Address: Finnegan, Henderson, Farabow, Garrett & Dunner, L.L.P., 1300 I Street, N.W., Washington, DC, 20005-3315, US

(21) Appl. No.: 09/731,787

(22) Filed: Dec. 8, 2000

(30) Foreign Application Priority Data
Dec. 9, 1999 (DE) 19959546.1-41

(51) Int. Cl. A61K 31/685; A61K 31/52; A61K 31/337

(52) U.S. Cl. 514/78; 514/261; 514/449; 514/680

(57) ABSTRACT

The present invention relates to a pharmaceutical composition comprising at least one active compound having cytostatic activity, at least one biological electron acceptor and customary pharmaceutical additives, and to its use for the treatment of tumor diseases, in particular for the treatment of cancer.

The present invention relates to a pharmaceutical composition comprising at least one active compound having cytostatic activity, at least one active biological electron acceptor and customary pharmaceutical additives, and to its use for the treatment of tumor diseases, in particular for the treatment of cancer.

A number of pharmaceutical compositions are known for the treatment of tumor diseases. These known, and in some cases also already used, cytostatics differ in the fact that they contain different cytostatically active compounds, such as, in particular, taxane, taxane derivatives, taxols, quinones, benzoquinones, other quinones and also derivatives and/or salts of these compounds.

Particularly promising cytostatics are 4H-1-benzopyran-4-one and its derivatives, which are also called flavopiridols and are disclosed in European Patent Application 0 137 193, in European Patent 0 366 061 and German Offenlegungsschrift 36 12 337. Especially promising are those compounds which are described in European Patent 0 366 061 as compounds of the formula B

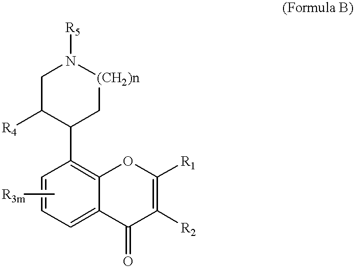

wherein

R₁ is hydrogen, alkyl having 1 to 6 carbon atoms, aryl-C₁-C₄-alkyl, substituted C₁-C₆-alkyl, C₃-C₆-cycloalkyl, a C₃-C₉-heterocycle having 1, 2 or 3 heteroatoms such as N, S, O or any combinations thereof, C₃-C₆-cycloalkyl-C₁-C₄-alkyl, C₂-C₆-alkenyl, C₂-C₆-alkynyl, aryl, polycyclic radicals including aromatic heterocyclic radicals, substituted aryl, carboxyl or an aldehyde or COO-C₁-C₄-alkyl group, a primary amino, alkylamino, aralkylamino, dialkylamino, amido, arylamino or diarylamino group or —CH₂O—C₁-C₄-alkyl;

R₂ is hydrogen or C₁-C₃-alkyl;

R₃ is hydroxyl or OCH₃;

R₄ is hydroxyl;

R₅ is CH₃;

m is equal to the number 2 and

n is equal to the number 1,

and their pharmacologically acceptable salts.

The compounds according to the invention have two asymmetric centers, one at the site of linkage of the nitrogen heterocyclic ring to the benzopyran moiety (C-4 min), the other at the carbon atom substituted by R4 (C-3 min), on account of which two pairs of optical isomers are possible. The definition of the compounds according to the invention includes all possible stereoisomers and their mixtures. Very particularly, it includes the racemic forms and the isolated optical isomers having the activity indicated. The two racemates can be separated by physical methods, such as, for example, fractional crystallization. The individual optical isomers can be obtained from the racemates by standard methods, such as, for example, salt formation with an optically active acid and subsequent crystallization.

Suitable alkyl groups for R₁ are, for example, straight-chain or branched radicals having up to 6, preferably up to 5, carbon atoms, e.g. methyl, ethyl, propyl, isopropyl, t-butyl, pentyl or isopentyl groups.

Suitable substituted alkyl groups for R₁ are, for example, haloalkyl, such as trifluoromethyl, hydroxyalkyl, such as hydroxyethyl, or carboxyalkyl, such as carboxyethyl.

Suitable examples of a cycloalkyl group as R₁ having 3 to 6 carbon atoms are cyclopropyl, cyclobutyl, cyclopentyl or cyclohexyl. Cyclopropylmethyl is an example of cycloalkylalkyl.

An example of an arylalkyl group as R₁ is a phenylalkyl group, in which the phenyl group is unsubstituted or mono- or polysubstituted by substituents such as halogen, C₁-C₄-alkyl, C₁-C₄-alkoxy, nitro or a trifluoromethyl group, amino group or substituted amino group.

An example of an aryl group as R₁ is a phenyl group which is unsubstituted or mono- or polysubstituted by substituents such as halogen, C₁-C₄-alkyl, C₁-C₄-alkoxy, hydroxyl, carboxyl, COO-alkyl, CONH₂, CONH-alkyl, CON-(alkyl)₂, nitro or trifluoromethyl, amino, C₁-C₄-alkylamino, di-C₁-C₄-alkylamino, aromatic heterocycles such as pyridyl groups and polycyclic aromatic radicals such as naphthyl groups.

A suitable example of an alkylamino group as R₁ is (CH₂)ₙ—NR₆R₇, wherein n is equal to 1 to 3, and R₆ and R₇ are alkyl and have the same meaning as indicated above for alkyl R₁ to R₅; moreover, R₆ and R₇, together with the nitrogen atom to which they are bonded, can be a heterocyclic ring having one or more heteroatoms. Suitable examples of heterocyclic rings which are formed by R₆ and R₇ and the nitrogen to which they are bonded, are piperidine, pyrrolidine, morpholine, piperazine or imidazole, which can be unsubstituted or substituted in one or more positions by C₁-C₄-alkyl, C₁-C₄-alkoxy, aryl or a hydroxyl or amino group.

Suitable examples of salts of the compounds according to the invention with inorganic or organic acids are the chloride, bromide, sulfate, phosphate, acetate, oxalate, tartrate, citrate, maleate or fumarate.

A significant and serious disadvantage of the known pharmaceutical compositions employed for the treatment of tumor diseases lies in the fact that compositions of this type in some cases cause severe side effects in the patient, where the nature and intensity of these side effects can vary appreciably, depending on the cytostatic present in each case, its concentration and its chemical structure.

Thus EP 0 542 807, for example, discloses suppression of the undesired side effects of the cytostatic cisplatin by the presence or simultaneous administration of 2-phenyl-1,2-benzisoselenazol-3(2H)-one (Ebselen).

The present invention is based on the object of making available a pharmaceutical composition for the treatment of tumor diseases, in particular for the treatment of cancer, with which the side effects caused by the cytostatic active compound are particularly effectively reduced and/or suppressed, even on long-term use.

This object is achieved according to the invention by a pharmaceutical composition having the characterizing features of patent claim 1.

According to the invention, a pharmaceutical composition for the treatment of tumor diseases, in particular for the treatment of cancer, is thus proposed wherein the composition according to the invention contains at least one active compound having cytostatic activity, in particular an active compound based on 4H-1-benzopyran-4-one, taxane, quinone, benzoquinone, anthraquinone and their derivatives and/or their salts, and also customary additives. In addition, at least one biological electron acceptor is present in the composition according to the invention.

The pharmaceutical composition according to the invention is based on the basic knowledge that active compounds having cytostatic activity, which serve for the control of tumors and in particular of cancer, accept energy-rich electrons from the mitochondrial membrane and then pass on these electrons to the oxygen present in the body or in the cells. By means of this, toxic oxygen free radicals are formed which also, expressed concisely, are called reactive oxygen species (ROSs). These energy-rich ROSs destroy the chemical structure of biomolecules, from which the body is built up, so that by means of this route very severe side effects can be caused.

Surprisingly, it has now been found that the previously mentioned excess and/or misdirected energy-rich electrons can be captured from the mitochondrial membrane of animal and/or human cells before these electrons can react with oxygen with formation of the toxic ROSs. Therefore, it is necessary that the pharmaceutical composition according to the invention contains appropriate biological electron acceptors, the at least one biological electron acceptor contained in the composition according to the invention preventing the excess and/or misdirected energy-rich electrons and thus the formation of ROSs as toxic metabolite. According to this explanation the pharmaceutical composition of the invention produces a markedly lowering of the side effects and thus an appreciable improvement in the tolerability of the cytostatic active compounds, so that when using the composition according to the invention for tumor treatment and in particular for cancer treatment, undesired side effects are completely or almost completely suppressed, even if the pharmaceutical composition according to the invention is administered over a long period of time. In addition, the biological electron acceptors contained in the pharmaceutical composition according to the invention can improve the solubility of certain active compounds having cytostatic activity or increase the absorbability of these active compounds such that additional advantages are made available by this means, as is explained in further detail below.

In other words, the pharmaceutical composition according to the invention thus contains at least one biological electron acceptor, this biological electron acceptor being defined by the fact that it is able, in the human and/or animal body, to capture the energy-rich electrons resulting or occurring during the administration of a cytostatic active compound such that the formation of ROSs is effectively suppressed. The biological electron acceptor itself is nontoxic.

A first, particularly advantageous embodiment of the pharmaceutical composition according to the invention comprises as biological electron acceptor at least one compound of the type which contains at least one functional group of the formula 1

—(CH₂)₂—N⁺—(CH₃)₃ (Formula 1).

For the biological electron acceptor which contains at least one functional group of the formula 1 represented above, it was surprisingly found that undesired energy-rich electrons which, as already explained above, are present in misdirected form or in an excess, are captured by the functional group represented in formula 1.

In the course of this capture reaction, two electrons and one proton are transferred in the form of a hydride ion —H⁻—to the functional group of the electron acceptor with elimination of methane. This leads to the hydride ion and thus the energy-rich electrons not being transferred to oxygen in the membranes of the mitochondria but being able to leave the body in the form of methane.

It was possible to demonstrate the fact that the capture reaction described beforehand also actually proceeds in this way if biological organisms are treated with the pharmaceutical composition according to the invention by means of experiments with liver cells and also in animal experiments, the formation of methane being detected as a result of excess energy-rich electrons of this type, which, however, were captured in the case of the preparation according to the invention. It was possible using this capture reaction to convert the toxic, excess energy into a gas which is indifferent and nontoxic for the human or animal body and which is exhaled by the lungs.

In particular, the pharmaceutical composition according to the invention contains as biological electron acceptor S-adenosylmethionine, a derivative and/or a salt thereof. The S-adenosylmethionine, its derivative and/or its salt-contained therein function as a biological electron acceptors in this embodiment of the invention by means of a methyl group that acts as an electron acceptor and thus captures excess energy-rich and/or misdirected electrons resulting during the administration of the cytostatic active compounds so that, accordingly, at least one methyl group is eliminated and converted into harmless methane. Such electrons therefore cannot lead to an adverse effect on and/or damage to cells as a main cause of the side effects occurring during the administration of cytostatics.

In a further embodiment of the pharmaceutical composition according to the invention, this contains a biological electron acceptor which is a natural electron acceptor present in aerobic cells. In particular, these electron acceptors are compounds of the formula A, below, which are isolated from biological material, preferably soybeans, corn, wheat, rape seed, oil-bearing fruits and oil-bearing seeds and/or eggs or which are prepared or derivatized synthetically or semisynthetically.

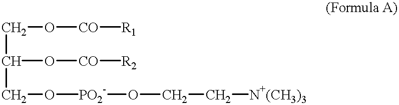

In formula A, R₁ and R₂ can be identical or different and are each hydrogen or the radical of a saturated or unsaturated C₁-C₂₂-fatty acid, preferably palmitic, stearic, oleic, linoleic or linolenic acid.

The synthetic or semisynthetic compounds and derivatives are preferably dipalmitoylphosphatidylcholine (DPPC), distearyl-phosphatidylcholine (DSPC) and dimyristoylphosphatidylcholine (DMPC).

The undesired formation of the ROSs and thus the occurrence of side effects is also markedly suppressed by those embodiments of the pharmaceutical composition according to the invention in which the preparation contains as biological electron acceptor betaine, acetylcholine, choline, glycerophosphocholine, phosphatidylcholine, lysophosphatidyl-choline, carnitine, acylcarnitine, sphingomyelins both as individual substances and as mixtures and/or derivatives.

Embodiments of the pharmaceutical composition according to the invention which are particularly suitable and have a high suppression capacity with respect to the side effects contain the biological electron acceptor and the active compound having cytostatic activity in a molar mass ratio of between 0.1:1 and 5:1, preferably in a molar mass ratio of between 0.5:1 and 2:1.

A particularly advantageous embodiment of the pharmaceutical composition according to the invention provides for the fact that the preparation containing as biological electron acceptor a mixture of betaine with at least one fatty acid salt, the fatty acid salt preferably containing a main carbon chain, in particular a saturated and/or unsaturated main carbon chain, having 12 to 18 carbon atoms.

Apart from this previously mentioned mixture of betaine with the fatty acid salt, the pharmaceutical composition according to the invention can contain, also as a biological electron acceptor, at least one fatty acid salt, wherein the fatty acid salt preferably contains a main carbon chain, in particular a saturated and/or unsaturated main carbon chain, having 12 to 18 carbon atoms.

In particular, a betaine laurate, a betaine myristate, a betaine palmitate, a betaine stearate, a betaine oleate and/or a betaine linoleate are present as the fatty acid salt of the betaine in the previously described embodiment of the preparation according to the invention.

An embodiment of the pharmaceutical composition according to the invention which is particularly suitable and advantageous to use and also stable on storage proposes in this case the preparation according to the invention containing as a biological electron acceptor a phospholipid, in particular a plant phospholipid and preferably a soybean phospholipid. In this case, these phospholipid biological electron acceptors on the one hand very effectively suppress the side effects and on the other hand make it possible that those cytostatic active compounds which are poorly soluble can be appreciably better dissolved or stably dispersed or stably emulsified in a suitable nontoxic solvent. Moreover, it is possible by means of the use of phospholipids of this type as biological electron acceptors to prepare emulsions, nanoemulsions, liposomal formulations, mixed micelle-containing formulations or even the formation of complexes between the phospholipids and the active compounds having cytostatic activity, so that, accordingly, formulations of this type which contain the phospholipid biological electron acceptor and also the at least one active compound having cytostatic activity have a number of further advantages. These are expressed, for example, by the fact that the active compound having cytostatic activity is better and/or more readily dissolvable, dispersible or emulsifiable, on account of which, for example, the administration of liquid preparations is facilitated, that the storage stability is increased, that sterile filterability is afforded, that transparency is guaranteed or that the active compound is additionally encapsulated in an appropriate phospholipid vesicle, on account of which a greater concentration of active compound can be administered more rapidly and with a higher degree of efficacy.

The previously mentioned advantages in particular occur if the pharmaceutical composition according to the invention contains as active compound having cytostatic activity 4H-1-benzopyran-4-one and/or a derivative or salt thereof, i.e. in particular the flavopiridol HCl described as a preferred compound in European Patent 0 366 061, flavopiridol being described by the formula C:

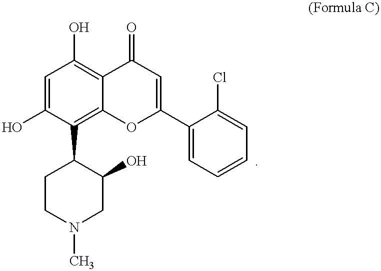

Surprisingly, it was possible to determine that those preparations which as biological electron acceptor contain phospholipids, in particular the specific phospholipids mentioned previously or those additionally described below, have an outstanding storage stability with retention of the cytostatic activity, although it was to be feared that in particular in the case of those embodiments which contain phosphatidylcholine as a biological electron acceptor and an active compound based on 4H-1-benzopyran-4-one, its derivatives and/or salts and preferably flavopiridol, during storage they exhibit an undesired interaction between the biological electron acceptor and the active compound, which would have led to a deactivation and/or to an undesired modification of the active compound and/or of the electron acceptor.

Particularly suitable embodiments are those phospholipid biological electron acceptors in which the phospholipid, in particular the phospholipid isolated from soybeans, contains a concentration of phosphatidylcholine of at least 50% by weight, based on the total amount of the electron acceptor contained in the composition. To be mentioned as particularly suitable here are, for example, those phospholipid biological electron acceptors which in addition to at least 50% by weight of phosphatidylcholine contain a liquid vehicle system, in particular a pharmaceutically acceptable, primary C₂-C₄-alcohol and/or a natural oil and/or a polyalkylene glycol. With respect to the oily components in liquid phosphatidylcholine preparations of this type, it is to be stressed that for this purpose, in particular, liquid triglycerides, as for example caprylic acid/capric acid triglycerides, glyceryl stearates, ascorbyl palmitates, oleyl palmitates, coconut oil, polyethylene glycol and/or polyethylene glycol, in each case alone or as a mixture, are prefered, where the concentration of phosphatidylcholine in oily preparations of this type then preferably varies between 45% by weight and 75% by weight, in particular between 50% by weight and 60% by weight.

If, on the other hand, a higher amount of energy-rich electrons and/or toxic oxygen free radicals (ROSs) are to be captured in the pharmaceutical composition according to the invention, then as phospholipid biological electron acceptors those phospholipid compositions are used whose concentration of phosphatidylcholine is greater than 70% by weight and preferably greater than 80% by weight and in particular greater than 90% by weight, meaning the concentration of phosphatidylcholin in relation to the total amount of the phospholipid electron acceptor contained in the preparation. Thus, in particular, the preparation according to the invention can contain a phospholipid biological electron acceptor of the type which is formulated as a liquid and which contains between 70 and 80% by weight of phosphatidylcholine in addition to the previously mentioned oily substances. Highly pure phospholipid biological electron acceptors then contain between 90% by weight and 96% by weight of phosphatidylcholine, based on the amount of the phospholipid biological electron acceptor.

A further, particularly advantageous embodiment of the pharmaceutical composition according to the invention proposes the previously represented phospholipid biological electron acceptor, in addition to the abovementioned concentration of phosphatidylcholine, containing at least additionally one negatively charged phospholipid, in particular N-acylphosphatidylethanolamine, phosphatidylinositol, phosphatidylglycerol, phosphatidic acid and also salts and/or derivatives of the previously mentioned negatively charged phospholipids. In this case, these negatively charged phospholipids cause corresponding liquid formulations of the pharmaceutical composition according to the invention to have a high transparency, even if the active compound having cytostatic activity is not soluble in the solvent used in each case, and the storage stability to be correspondingly increased so that no bottom sediment is formed even with an extremely long storage time.

Preferably, the concentration of the negatively charged phospholipids in the previously described embodiment of the preparation according to the invention varies between 2% and 10% by weight, based on the total amount of the phospholipid biological electron acceptor contained in the preparation.

Above, in connection with the phospholipid biological electron acceptor, it has been described that the electron acceptor comprises a phospholipid or a phospholipid mixture, in particular also phosphatidylcholine. Among these, in addition to the already repeatedly mentioned 1,2-diacylglycero-3-phosphocholine [(3-sn-phosphatidyl)choline], 1,2-diacylglycero-3-phosphoethanolamine, 1,2-diacylglycero-3-phosphoinositol, 1,2-diacylglycero-3-phosphoserine, 1,2-diacylglycero-3-phosphoglycerol and 1,2-diacylglycerol-3-phosphate are also preferably included, in each case alone or as a mixture.

In a particularly suitable embodiment of the previously described preparations according to the invention, this contains a phosphatidylcholine of the type in which the acyl radicals contained in the phosphatidylcholine consist to

61-73% by weight of the linoleic acid radical,

10-14% by weight of the palmitic acid radical,

8-12% by weight of the oleic acid radical,

4-6% by weight of the linolenic acid radical,

3-5% by weight of the stearic acid radical and

up to 2% by weight of other fatty acid radicals.

As already mentioned above, the phospholipid provided in the preparation according to the invention can be a phospholipid mixture. Suitable phospholipids for this are, in particular, 1,2-diacylglycero-3-phosphate (1,2-diacylglycero-3-phosphoethanolamine, 1,2-diacylglycero-3-phosphoinositol, 1,2-diacylglycero-3-phosphoserine, 1,2-diacylglycero-3-phosphoglycerol and/or 1,2-diacylglycero-3-phosphate), preferably up to 30% by weight of the abovementioned 1,2-diacylglycero-3-phosphates being contained in the phospholipid mixture, while an embodiment of the preparation according to the invention of this type then contains at least 70% by weight of the 1,2-diacylglycero-3-phosphocholine. In this case, the previously mentioned percentage mass details relate to the total mass of the phospholipid biological electron acceptor contained in the preparation according to the invention.

Another, particularly suitable embodiment of the preparation according to the invention includes as phospholipid a 1,2-diacylglycero-3-phosphocholine, in which the 1-acyl radical comprises

45-61% by weight of linoleic acid radicals,

19-26% by weight of palmitic acid radicals,

8-12% by weight of oleic acid radicals,

4-6% by weight of linolenic acid radicals,

6-9% by weight of stearic acid radicals and

2% by weight of other fatty acid radicals,

while the 2-acyl radical consists to

77-85% by weight of the linolenic acid radical,

1-2% by weight of the palmitic acid radical,

8-12% by weight of the oleic acid radical,

4-6% by weight of the linolenic acid radical,

0-1% by weight of the stearic acid radical and

2% by weight of other fatty acid radicals.

With respect to the respective formulation of the pharmaceutical composition according to the invention, it is to be stressed that in this case any formulation which allows oral, parenteral and/or topical administration of the preparation according to the invention is suitable. Accordingly, the preparation according to the invention is prepared as a tablet, capsule, solution, emulsion, dispersion, liposome system and/or as a liquid mixed micelle system.

Sugar-coated formulations and sugar-coated delayed-release formulations are also included in the scope of the invention. Acid-resistant and enteric formulations are preferred. Pharmaceutically customary additives comprise enteric coatings such as cellulose acetate phthalate, polyvinyl acetate phthalate, hydroxypropylmethylcellulose phthalate and anionic polymers of methacrylic acid and methyl methacrylate.

Suitable pharmaceutical compounds for oral administration can be present in separate units, such as, for example, capsules, cachets, sucking tablets or tablets, as powders or granules; as a solution or suspension in an aqueous or nonaqueous liquid; or as an oil-in-water or water-in-oil emulsion. These compositions can be prepared by any suitable pharmaceutical method which comprises a step in which the active compound and the carrier (which can consist of one or more additional constituents) are brought into contact. In general, the compositions are prepared by uniform and homogeneous mixing of the active compound with a liquid and/or finely divided solid carrier, after which the product, if necessary, is shaped. Thus it is possible, for example, to prepare a tablet by pressing or shaping a powder or granules of the compound, if appropriate with one or more additional constituents. Pressed tablets can be produced by tableting the compound in free-flowing form, such as, for example, a powder or granules, if appropriately mixed with a pharmaceutically customary additive such as a binder, a lubricant, an inert diluent and/or one or more surface-active agents/dispersants in a suitable machine. Shaped tablets can be produced by shaping the powdered compound, moistened with an inert liquid diluent, in a suitable machine.

Pharmaceutical compositions which are suitable for peroral (sublingual) administration comprise sucking tablets, which as pharmaceutically tolerable additives customarily contain sucrose and gum arabic or tragacanth, and pastilles, which comprise the compound in an inert base such as gelatin and glycerol or sucrose and gum arabic.

Oral and peroral preparations can optionally contain further pharmaceutically customary additives, for example a flavoring, in particular a fruit flavor, or sweeteners, for example saccharin sodium.

Suitable pharmaceutical compositions for parenteral administration preferably comprise sterile aqueous preparations which are preferably isotonic with the blood of the intended recipient. These preparations are preferably administered intravenously, although administration can also take place subcutaneously, intramuscularly or intradermally as an injection. These preparations can preferably be produced by mixing the compound with water and rendering the solution obtained sterile and isotonic with the blood.

Suitable pharmaceutical compositions for topical application to the skin are preferably present as an ointment, cream, lotion, paste, spray, aerosol or oil. Pharmaceutically customary additives which can be used are vehicles such as, for example, petroleum jelly, lanolin, polyethylene glycols, alcohols and combinations of two or more of these substances. Transdermal administration is also possible. Suitable pharmaceutical compositions for transdermal administrations can be present as individual patches, which are suitable for a long-term close contact with the epidermis of the patient. Such patches suitably contain active compound and electron acceptor in an optionally buffered aqueous solution, dissolved and/or dispersed in an adhesive or dispersed in a polymer. As a particular possibility, the active compound, such as described, for example, in Pharmaceutical Research, 2(6): 318 (1986), can be released by electrotransport or ionophoresis.

An embodiment of the preparation according to the invention which is particularly suitable and relatively simple to administer provides in this case for the preparation being formulated as an injection or infusion fluid, this preparation then preferably containing as active compound having cytostatic activity flavopiridol-HCl, doxorubicin-HCl, idarubicin-HCl and/or daunorubicin-HCl. The previously mentioned active compounds are then dissolved, dispersed, emulsified and/or prepared in the form of liposomes and/or mixed micelles in a suitable solvent, i.e., for example, water, ethanol, propanol, isopropanol and/or mixtures thereof. In addition, the previously mentioned oils and/or polyalkylene oxides can be present in these liquid preparations. These liquid administration forms furthermore contain as biological electron acceptor betaine dihydrogencitrate, choline citrate, phospholipids, preferably the actual phospholipids mentioned previously and in particular phosphatidylcholine, and/or ademethionine tosylate bis(sulfate).

With respect to the concentration of the cytostatic active compound in the previously described liquid administration forms, it is to be emphasized that this concentration depends on the particular solvent selected and the solubility, the dispersibility or the emulsifiability of the particular cytostatic active compound in this solvent. Concentrations of the cytostatic active compound which have proven particularly suitable here are those which vary between 1 mg and 200 mg, in particular between 5 mg and 40 mg.

Depending on the concentration of the cytostatic active compound and the chemical structure thereof, the concentration of biological electron acceptor in the pharmaceutical composition according to the invention is also defined. It has been shown here that the preparation according to the invention, in the case of a liquid formulation, preferably contains concentrations of biological electron acceptor of between 50 mg and 3 g, in particular between 250 mg and 1 g, the nature of the biological electron acceptor used in each case, i.e. its chemical structure, also having an influence on the concentration of the biological electron acceptor to be employed in each case.

If, however, the pharmaceutical composition according to the invention is formulated as a composition for oral administration, i.e. in particular as a tablet, granules or powder, the composition according to the invention preferably contains as active compound having cytostatic activity flavopiridol-HCl and/or idarubicin-HCl, while the biological electron acceptor provided is betaine dihydrogencitrate, choline citrate, phospholipids and in particular phosphatidylcholine and/or ademethionine tosylate bis (sulfate).

As already explained in the case of the previously described liquid compositions, the concentration of the cytostatic active compound in the formulations to be administered orally depends on the particular active compound or active compound mixture selected in each case, preferred concentrations of the cytostatic active compound varying between 5 mg and 70 mg, in particular between 15 mg and 40 mg.

In particular, the previously described oral administration forms contain concentrations of biological electron acceptor which are between 50 mg and 3 mg, preferably between 250 mg and 1 mg.

Previously, embodiments were described for the pharmaceutical composition according to the invention in which the composition according to the invention simultaneously contains at least one active compound having cytostatic activity and additionally also the biological electron acceptor.

Another, particularly suitable embodiment of the invention, comprises two liquid or two solid preparations or one solid and one liquid preparation, a first preparation containing at least one active compound having cytostatic activity based on 4H-1-benzopyran-4-ones, taxane, quinone, benzoquinone, anthraquinone, their derivatives and/or their salts, and customary additives, while a second preparation then contains the biological electron acceptor. In other words, in this embodiment of the pharmaceutical composition according to the invention, the first preparation is formulated separately from the second preparation, such that, in particular, if these two preparations are administered in liquid or powder form an individual dose of the cytostatic active compound can be given and additionally, previously, simultaneously or thereafter, a concentration of the biological electron acceptor chosen depending on the reaction of the patient to be treated and individually tailored thereto is made available. In this embodiment of the invention, the embodiments that have been previously described for the embodiments of the preparation according to the invention, wherein both active compound and biological electron acceptor are formulated in one formulation, correspondingly apply.

The present invention in particular also relates to the use of the previously described pharmaceutical composition for the treatment of tumors, in particular for the treatment of cancer, the preparation according to the invention being administered in a daily dose of between 0.0001 g and 2 g, in particular between 0.01 g and 1 g, of the cytostatic active compound and in a daily dose of between 0.1 g and 100 g, in particular between 5 g and 50 g, of the biological electron acceptor, the previously mentioned dose rates in each case relating to a square meter of the body surface of the patient to be treated.

The composition according to the invention is explained in greater detail below with the aid of exemplary embodiments without, however, being restricted to these.

### EXEMPLARY EMBODIMENTS A1 to A5

The following Exemplary Embodiments A1 to A5 relate to those preparations which are intended for parenteral administration and which contain different active compounds having cytostatic activity and different biological electron acceptors.

For the production of the preparations A1, A2 and A5, the constituents mentioned in Table 1 were in each case dissolved in ethanol in the amounts mentioned there. After stripping off the solvent under vacuum and inert gas, the residue which remained was dispersed in 20 l (Preparation A1 and A5) or 10 l (Preparation A2) of water. The dispersion was then homogenized with formation of liposomes having a mean particle diameter of between 0.1 μm and 1 μm.

In the case of the preparations A1 and A5, in each case 2 kg of maltose, dissolved in 2 l of water, were added to the homogenized dispersions and in the case of the preparation A2 1 kg of maltose, dissolved in 1 l of water, the relevant dispersion again being homogenized.

Liposomal formulations having a mean liposome diameter of between 0.1 μm and 1 μm resulted here.

The homogeneous mixtures were sterile filtered using a filter having a pore size of 0.2 μm.

The preparations sterile filtered in this way were dispensed into vials, each vial of the preparation A1 containing 100 mg of flavopiridol in 20 ml, the preparation A2 10 mg of doxorubicin in 20 ml and the preparation A5 100 mg of flavopiridol in 20 ml.

For storage, the relevant filled vials were subsequently freeze dried.

For infusion, each vial is then redispersed with 20 ml of water and mixed after addition of 250 ml of glucose solution (glucose concentration: 5% by weight).

TABLE 1

| | A1 | A2 | A5 |
|---|---|---|---|
| Flavopiridol-HCl | 100 g | — | 100 g |
| Doxorubicin | — | 10 g | — |
| Phosphatidylcholine | 2 kg | 1 kg | 2 kg |
| DSPG | 40 g | 20 g | 40 g |
| Betaine linoleate | — | — | 250 g |
| Ethanol | 10 l | 5 l | 10 l |

DSPG = Distearoylphosphatidylglycerol
Phosphatidylcholine = Phospholipon 90; phospholipid
concentration: 93 ± 3% by weight

For the production of the preparations A3 and A4, the constituents mentioned in Table 2 were in each case dissolved in water in the amounts mentioned there. After this, the solutions were sterile filtered using a filter having a pore size of 0.2 μm. After dispensing the sterile filtered solution having a concentration of 10 mg of the active compound having cytostatic activity into 10 ml vials, the vials were freeze dried.

Immediately before infusion, the contents freeze dried in this way were redispersed with 10 ml of water and mixed with 200 ml of glucose solution (glucose concentration: 5% by weight).

TABLE 2

| | A3 | A4 |
|---|---|---|
| Doxorubicin | — | 10 g |
| Idarubicin | 10 g | — |
| Betaine dihydrogencitrate | 200 g | — |
| Choline citrate | — | 250 g |
| Lactose | 200 g | 250 g |
| Water | 10 l | 10 l |

### EXEMPLARY EMBODIMENTS B1 to B5

The following Exemplary Embodiments B1 to B5 relate to those preparations which are intended for parenteral administration and in which the different active compounds having cytostatic activity and the different biological electron acceptors are prepared and stored separately from one another, such that the first preparation containing the active compound having cytostatic activity is mixed with the second preparation containing the biological electron acceptor only immediately before parenteral administration.

For the production of the first preparation which contains the active compound having cytostatic activity, the constituents mentioned in Table 3 were dissolved in the amounts mentioned there. After this, the solution was sterile filtered using a 0.2 μm filter, in the case of the preparation B1 1 mg of fluorouracil being dispensed into 40 ml ampoules, in the case of the preparation B2 20 mg of daunorubicin being dispensed into 20 ml vials, in the case of the preparation B3 10 mg of doxorubicin being dispensed into 5 ml vials, in the case of the preparation 34 10 mg of idarubicin being dispensed into 5 ml vials and in the case of the preparation B5 10 mg of mitomycin being dispensed into 10 ml vials.

The vials filled with the preparations B2 to B5 were freeze dried and appropriately stored.

For the preparation of the second preparations B1, B2, B4 and B5 which contain the biological electron acceptor, the constituents mentioned in Table 4 were dissolved in water in the amounts mentioned there. After this, the relevant solution was sterile filtered using a 0.2 μm filter.

In the case of the second preparation B1, 500 mg of betaine dihydrogencitrate were dispensed into 10 ml ampoules, in the case of the preparation B2 100 mg of choline citrate were dispensed into 5 ml ampoules, in the case of the preparation B4 200 mg of choline citrate were dispensed into 10 ml ampoules and in the case of the preparation B5 250 mg of betaine dihydrogencitrate were dispensed into 5 ml ampoules.

The second preparation 33 was prepared in such a way that 10 kg of phosphatidylcholine were dissolved in 20 l of ethanol together with 20 g of DSPG (distearoylphosphatidyl-glycerol). After this, the ethanol was stripped off under vacuum and inert gas. The residue was dispersed with 20 l of water and then homogenized with formation of a liposomal formulation, the liposome diameter varying between 0.1 μm and 1 μm.

A solution consisting of 10 kg of maltose and 10 l of water was then added to this liposomal formulation. Mixing was subsequently carried out until a transparent, homogeneous dispersion resulted.

The dispersion prepared in this way was sterile filtered using a 0.2 μm filter.

The sterile-filtered dispersion was dispensed into 20 ml vials containing 1 g of phosphatidylcholine. After this, the vials were freeze dried.

TABLE 3
Compositions B1 to B5 containing the active compound having
cytostatic activity

| | B1 | B2 | B3 | B4 | B5 |
|---|---|---|---|---|---|
| Fluorouracil | 1 g | — | — | — | — |
| Daunorubicin | — | 100 g | — | — | — |
| Doxorubicin | — | — | 100 g | — | — |
| Idarubicin | — | — | — | 100 g | — |
| Mitomycin | — | — | — | — | 10 g |
| Maltose | — | 200 g | — | — | — |
| Lactose | — | — | 10 kg | 10 kg | 100 g |
| Water | 10 l | 20 l | 50 l | 50 l | 10 l |

TABLE 4
Composition B1, B2, B4 and B5 containing the biological
electron acceptor

| | B1 | B2 | B4 | B5 |
|---|---|---|---|---|
| Betaine dihydrogen- | 500 g | — | — | 250 g |
| citrate | | | | |
| Choline citrate | — | 100 g | 200 g | — |
| Water | 10 l | 5 l | 10 l | 5 l |

Immediately before the infusion, an ampoule of the first preparation B1 was mixed with an ampoule of the second preparation B1 with addition of 250 ml of glucose solution (glucose concentration: 5% by weight).

Immediately before the infusion, a vial of the first preparation B2 was redispersed in 20 ml of water and mixed with an ampoule of the second preparation B2 with addition of 250 ml of glucose solution (glucose concentration: 5% by weight).

Immediately before the infusion, a vial of the first preparation B3 was redispersed with 5 ml of water and mixed with a vial of the second preparation B3, where the second preparation had previously been redispersed with 20 ml of water. For this, addition and mixing with 250 ml of glucose solution was additionally carried out (glucose concentration: 5% by weight).

Immediately before the infusion, a vial of the first preparation B4 was redispersed with 10 ml of water. For this, addition of an ampoule of the second preparation B4 was carried out, where previously addition and mixing with 200 ml of glucose solution (glucose concentration: 5% by weight) had also been carried out.

The first preparation B5 was redispersed with 10 ml of water immediately before the infusion and subsequently mixed with an ampoule of the second preparation B5 and 50 ml of glucose solution (glucose concentration: 5% by weight).

### EXEMPLARY EMBODIMENTS C1 to C4

The following Exemplary Embodiments C1 to C4 relate, like the previously described Exemplary Embodiments B1 to B5, to those preparations which are intended for parenteral administration and which contain, in a first preparation, different active compounds having cytostatic activity and, in a second preparation, different biological electron acceptors, the first preparation being mixed with the second preparation only immediately before use.

For the production of the first preparations C1 to C4 which contain the active compound having cytostatic activity, the constituents mentioned in Table 5 were in each case dissolved in ethanol in the amounts mentioned there. After stripping off the ethanol under vacuum and inert gas, the residue which remained was dispersed in water, 20 l of water being used in the case of the preparation C1 and 10 l of water in each case being used in the case of the preparations C2 to C4.

Homogenization of the dispersion prepared in this way was subsequently carried out.

After this, an aqueous maltose solution was added to the homogenized dispersion, in the case of the first preparation C1 this maltose solution containing 2 kg of maltose and 2 l of water and in the case of the preparations C2 to C4 this maltose solution in each case containing 1 kg of maltose and 1 l of water.

After homogeneous mixing, the dispersion thus resulting was sterile filtered using a 0.2 μm filter.

After dispensing 100 mg of flavopiridol into 20 ml vials (Cl), 10 mg of daunorubicin into 10 ml vials (C2), 10 mg of idarubicin into 10 ml vials (C3) and 10 mg of doxorubicin into 10 ml vials (C4), the relevant vials were freeze dried.

For the production of the second preparations C1 to C4 which contain different biological electron acceptors, the constituents mentioned in Table 6 were in each case dissolved in water in the amounts mentioned there. After this, the relevant solution was sterile filtered using a 0.2 μm filter and dispensed into ampoules, these ampoules containing 250 mg of betaine hydrogencitrate in 5 ml (C1), 100 mg of choline citrate in 5 ml (C2), 250 mg of choline citrate in 5 ml (C3) and 250 mg of betaine hydrogencitrate in 5 ml (C4).

TABLE 5
Preparations C1 to C4 containing the active compound having
cytostatic activity

| | C1 | C2 | C3 | C4 |
|---|---|---|---|---|
| Flavopiridol-HCl | 100 g | — | — | — |
| Phosphatidylcholine | 2 kg | 1 kg | 1 kg | 1 kg |
| Daunorubicin | 100 g | 10 g | — | — |
| Doxorubicin | — | — | — | 10 g |
| Idarubicin | — | — | 10 g | — |
| DSPG | 40 g | 20 g | 20 g | 20 g |
| Ethanol | 10 l | 5 l | 5 l | 50 l |

DSPG = Distearoylphosphatidylglycerol

TABLE 6
Preparations C1 to C4 containing the biological electron
acceptor

| | C1 | C2 | C3 | C4 |
|---|---|---|---|---|
| Betaine | 250 g | — | — | 250 g |
| dihydrogencitrate | | | | |
| Choline citrate | — | 100 g | 250 g | — |
| Water | 5 l | 5 l | 5 l | 5 l |

Immediately before the infusion, the respective dry cytostatic active compound stored in vials was redispersed in water, in the case of Exemplary Embodiment C1 20 ml of water being used and in the case of Exemplary Embodiments C2 to C4 10 ml of water in each case being used for this. After this, thorough mixing of the redispersed active compounds with the previously described second preparations C1 to C4, which were stored in corresponding ampoules, was carried out. Furthermore, 250 ml of glucose solution (glucose concentration: 5% by weight) were in each case added and mixed with the two liquids previously mentioned.

### EXEMPLARY EMBODIMENTS D1 to D4

The following Exemplary Embodiments D1 to D4 relate to those preparations which are intended for oral administration and which simultaneously contain different active compounds having cytostatic activity and different biological electron acceptors.

For the production of the sachets described in Exemplary Embodiments D1 to D3, the constituents mentioned in Table 7 were mixed homogeneously with one another in an appropriate mixing device.

After this, the corresponding sachet was dispensed at 100 mg, such that an idarubicin concentration of 25 mg/sachet accordingly resulted.

For use of a preparation of this type to be taken orally, it is then only necessary to disperse the sachet according to the composition D1 to D3 in a glass of water.

TABLE 7
Constituents of the compositions D1 to D3 to be
administered orally

| | D1 | D2 | D3 |
|---|---|---|---|
| Idarubicin | 25 kg | 25 kg | 25 kg |
| Betaine dihydrogencitrate | 500 kg | — | — |
| Choline citrate | — | 500 kg | 500 kg |
| Sorbitol | 200 kg | 200 kg | 200 kg |
| Mannitol | 250 kg | 250 kg | 250 kg |
| Sodium cyclamate | 10 kg | 10 kg | 10 kg |
| Lemon flavor | 15 kg | 15 kg | 15 kg |

The preparation D4 to be administered orally was prepared as follows:

25 kg of idarubicin were homogeneously mixed with 100 kg of microcrystalline cellulose and 5 kg of magnesium stearate. 130 mg of this mixture were dispensed into hard gelatin capsules, which corresponded to a concentration of 100 mg of idarubicin. A second preparation was prepared by dissolving 200 mg of betaine hydrogencitrate, 50 kg of sorbitol and 2.5 kg of saccharin sodium in 5000 l of water. After sterile filtration using a 0.2 μm filter, this solution was dispensed into 5 ml drinking ampules, each drinking ampoule containing a concentration of biological electron acceptor of 200 mg.

For use, Exemplary Embodiment D4 was administered in such a way that the respective patient had taken a hard gelatin capsule which contained the active compound having cytostatic activity, together with the contents of a drinking ampoule. The water used in the Exemplary Embodiments for the preparation thereof is water for injections (W.F.I.).

1. A composition comprising at least one active compound having cytostatic activity, at least one biological electron acceptor, and also pharmaceutically customary additives.

2. The composition as claimed in claim 1, wherein the active compound having cytostatic activity is an active compound based on 4H-1-benzopyran-4-one, taxane, quinone, benzoquinone, anthraquinone, their derivatives and/or their salts.

3. The composition as claimed in one of claims 1 and 2, wherein the composition contains as biological electron acceptor at least one compound of the type which contains at least one functional group of the formula 1
—(CH₂)₂—N⁺—(CH₃)₃ (Formula 1).

4. The composition as claimed in one of claims 1 to 3, wherein the composition contains as biological electron acceptor at least one compound of the formula A

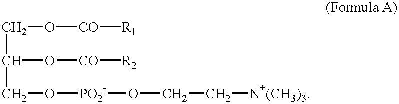

5. The composition as claimed in one of claims 1 to 4, wherein the composition contains as biological electron acceptor S-adenosylmethionine, a derivative and/or a salt thereof.

6. The composition as claimed in one of claims 1 to 5, wherein the biological electron acceptor is a natural electron acceptor present in aerobic cells.

7. The composition as claimed in one of claims 1 to 6, wherein the composition contains as biological electron acceptor betaine, acetylcholine, choline, glycerophosphocholine, phosphatidylcholine, lysophosphatidylcholine, carnitine, acylcarnitine, sphingomyelins, mixtures and/or derivatives thereof.

8. The composition as claimed in one of claims 1 to 7, wherein the molar mass ratio of the biological electron acceptor to the active compound having cytostatic activity varies between 0.1:1 and 5:1, preferably between 0.5:1 and 2:1.

9. The composition as claimed in one of claims 1 to 8, wherein the composition contains as biological electron acceptor a mixture of betaine with at least one fatty acid salt.

10. The composition as claimed in one of claims 1 to 9, wherein the composition contains as biological electron acceptor at least one fatty acid salt of the betaine.

11. The composition as claimed in one of claims 9 and 10, wherein the fatty acid salt contains a main carbon chain having 12 to 18 carbon atoms.

12. The composition as claimed in one of claims 9 to 11, wherein a betaine laurate, a betaine myristate, a betaine palmitate, a betaine stearate, a betaine oleate and/or a betaine linoleate is present in the composition as the fatty acid salt of the betaine.

13. The composition as claimed in one of claims 1 to 12, wherein the composition contains as biological electron acceptor a phospholipid, in particular a plant phospholipid and preferably soybean phospholipid.

14. The composition as claimed in claim 13, wherein the phospholipid contains a concentration of phosphatidylcholine of at least 50% by weight, based on the total amount of the phospholipid biological electron acceptor contained in the composition.

15. The composition as claimed in one of claims 13 and 14, wherein the concentration of the phosphatidylcholine is greater than 70% by weight, preferably greater than 80% by weight and in particular greater than 90% by weight, in each case based on the total amount of the phospholipid biological electron acceptor contained in the composition.

16. The composition as claimed in one of claims 13 to 15, wherein the phospholipid biological electron acceptor is a mixture of phospholipids, the mixture containing, in addition to phosphatidylcholine, at least additionally one negatively charged phospholipid, in particular phosphatidic acid.

17. The composition as claimed in claim 16, wherein the negatively charged phospholipid in the phospholipid mixture is present in a concentration of between 2% by weight and 10% by weight, based on the total amount of the phospholipid biological electron acceptor contained in the composition.

18. The composition as claimed in one of claims 1 to 17, wherein the composition is formulated as an injection or infusion fluid, wherein the composition contains as active compound having cytostatic activity flavopiridol-HCl, doxorubicin-HCl, idarubicin-HCl and/or daunorubicin-HCl and wherein the composition contains as biological electron acceptor betaine dihydrogencitrate, choline citrate, phosphatidylcholine and/or ademethionine tosylate bis(sulfate).

19. The composition as claimed in claim 18, wherein the concentration of the cytostatic active compound varies between 1 mg and 200 mg, in particular between 5 mg and 40 mg.

20. The composition as claimed in one of claims 18 and 19, wherein the concentration of biological electron acceptor varies between 50 mg and 3 g, in particular between 250 mg and 1 g.

21. The composition as claimed in one of claims 1 to 17, wherein the composition is formulated as a composition for oral administration, wherein the composition contains as active compound having cytostatic activity flavopiridol-HCl and/or idarubicin-HCl and wherein the composition contains as biological electron acceptor betaine dihydrogencitrate, choline citrate, phosphatidylcholine and/or ademethionine tosylate bis(sulfate).

22. The composition as claimed in claim 21, wherein the concentration of the cytostatic active compound varies between 5 mg and 70 mg, in particular between 15 mg and 40 mg.

23. The composition as claimed in one of claims 21 and 22, wherein the concentration of biological electron acceptor varies between 50 mg and 3 g, in particular between 250 mg and 1 g.

24. The composition as claimed in one of claims 1 to 23, wherein the composition comprises two liquid or solid compositions or one solid and one liquid composition, a first composition containing at least one active compound having cytostatic activity based on 4H-1-benzopyran-4-one, taxane, quinone, benzoquinone, anthraquinone, their derivatives and/or their salts and a second composition containing at least one biological electron acceptor.

25. The use of a composition as claimed in one of claims 1 to 24 for the production of a medicament for the treatment of tumor diseases, in particular for the treatment of cancer.

26. The use of the composition as claimed in claim 25, the daily dose of the cytostatic active compound being from 0.0001 9 to 2 g, in particular from 0.01 g to 1 g, the dose of the biological electron acceptor being from 0.1 g to 100 g, in particular from 5 g to 50 g, in each case based on a square meter of the body surface of the patient to be treated.
